(19) United States
(12) Patent Application Publication
(10) Pub. No.: US 2012/0035211 A1
(43) Pub. Date: Feb. 9, 2012

(54) NOVEL POLYMORPHS OF SAQUINAVIR

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara reddy, Hyderabad (IN); Kesireddy Subash Chander Reddy, Hyderabad (IN); Bhimireddy Srinivasa Reddy, Hyderabad (IN)

(73) Assignee: HETERO RESEARCH FOUNDATION, Hyderabad, Andhrapradesh (IN)

(21) Appl. No.: 13/122,011

(22) PCT Filed: Feb. 17, 2009
(86) PCT No.: PCT/IN2009/000109

§ 371 Date: May 10, 2011

(51) Int. Cl. A61K 31/4725; C07D 401/12

(52) U.S. Cl. 514/307; 546/146

(57) ABSTRACT

The present invention provides novel polymorphs of saquinavir, processes for their preparation and pharmaceutical compositions comprising them. The present invention also provides a process for purification of saquinavir. The present invention further provides a novel process for preparation of known saquinavir crystalline form I.

### FIELD OF THE INVENTION

The present invention provides novel polymorphs of saquinavir, processes for their preparation and pharmaceutical compositions comprising them. The present invention also provides a process for purification of saquinavir. The present invention further provides a novel process for preparation of known saquinavir crystalline form I.

### BACKGROUND OF THE INVENTION

Inhibitors of human immunodeficiency virus (HIV) protease have been approved for use in the treatment of HIV infection for several years. A particularly effective HIV protease inhibitor is (2S)—N′-[(1S,2R)-3-[(3S,4aS,8aS)-3-[[(1,1-dimethylethyl)amino]carbonyl]octahydro-2(1H)-isoquinolinyl]-2-hydroxy-1-(phenylmethyl)propyl]-2-[(2-quinolinylcarbonyl)amino]butanediamide, also known as saquinavir and its pharmaceutically acceptable salts such as saquinavir mesylate. Saquinavir and its pharmaceutically acceptable salts can be used as medicaments for the treatment of prophylaxis of viral infections in mammals, humans or non-humans. Saquinavir is represented by the following structure.

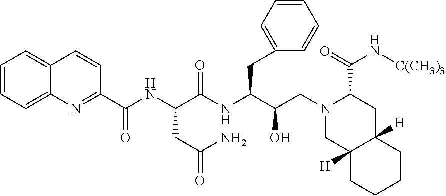

Polymorphism is defined as “the ability of a substance to exist as two or more crystalline phases that have different arrangement and/or conformations of the molecules in the crystal Lattice. Thus, in the strict sense, polymorphs are different crystalline forms of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning calorimetry (DSC) and Infrared spectrometry (IR).

Solvent medium and mode of crystallization play very important role in obtaining a crystalline form over the other.

Saquinavir can exist in different polymorphic forms, which differ from each other in terms of stability, physical properties, spectral data and methods of preparation.

U.S. Pat. No. 5,196,438 disclosed benzyloxycarbonyl- and 2-quinolylcarbonyl-amino acid derivatives and pharmaceutically acceptable acid addition salts thereof. Processes for the preparations of saquinavir and related compounds were disclosed in U.S. Pat. No. 5,196,438. According to U.S. Pat. No. 5,196,438, saquinavir is prepared by reacting N-(2-quinolylcabonyl)-L-asparagine with 2-[3(S)-amino-2(R)-hydroxy-4-phenylbutyl]-N-tert.butyl-deca hydro-(4aS,8aS)-isoquinoline-3(S)-carboxamide in tetrahydrofuran in presence of 3-hydroxy-1,2,3-benzotriazin-4(3H)-one and dicyclohexylcarbodiimide to give saquinavir.

Processes for the preparations of saquinavir were disclosed in the U.S. Pat. No. 5,451,678. According to U.S. Pat. No. 5,451,678, crystalline solid of saquinavir was obtained by chromatographing saquinavir residue obtained as the part of the reaction between N-(2-quinolylcabonyl)-L-asparagine and 2-[3(S)-amino-2(R)-hydroxy-4-phenylbutyl]-N-tert.butyl-decahydro-(4aS,8aS)-isoquinoline-3(S)-carboxamide on silica gel using 4% (by volume) methanol in dichloromethane for the elution. The crystalline saquinavir obtained by the process of the prior art is herein after designated as saquinavir crystalline form I. The powdered x-ray diffractogram (PXRD) of crystalline Form I is shown in FIG. 4. Crystalline Form I is characterized by peaks in the powder x-ray diffraction spectrum having 2θ angle positions at about 4.7, 5.3, 9.2, 16.1 and 18.1±0.2 degrees.

Processes for the preparations of saquinavir mesylate were disclosed in U.S. Pat. No. 5,750,649. According to U.S. Pat. No. 5,750,649, saquinavir mesylate was prepared by reacting saquinavir with methanesulphonic acid in ethyl acetate to give saquinavir mesylate.

WO Patent Publication No. 2006/134612 disclosed a process for the preparation of saquinavir mesylate.

We have discovered that saquinavir can be prepared in two well-defined and consistently reproducible crystalline forms and one amorphous form.

One object of the present invention is to provide a novel crystalline forms of saquinavir, process for their preparation and pharmaceutical compositions comprising them.

Another object of the present invention is to provide a process for purification of saquinavir.

Another object of the present invention is to provide a novel amorphous form of saquinavir and a process for preparing it and pharmaceutical compositions comprising it.

Still another object of the present invention is to provide a process for preparing saquinavir crystalline form I.

### SUMMARY OF THE INVENTION

In accordance with one aspect of the present invention, there is provided a novel crystalline form of saquinavir designated as form II characterized by peaks in the powder x-ray diffraction spectrum having 2θ angle positions at about 6.6, 11.3, 17.1, 18.3, 20.7 and 23.5±0.2 degrees.

In accordance with another aspect of the present invention, there is provided a process for the preparation of saquinavir crystalline form II, which comprises:
a) stirring saquinavir in a solvent system comprising an alcohol and water; and optionally in the presence of acetonitrile; and
b) isolating saquinavir crystalline form II.

In accordance with another aspect of the present invention, there is provided a process for purification of saquinavir which comprises:
a) stirring saquinavir in a solvent system comprising an alcohol, water and acetonitrile; and
b) isolating saquinavir to obtain substantially pure saquinavir.

In accordance with another aspect of the present invention, there is provided a novel crystalline form of saquinavir designated as form III characterized by peaks in the powder x-ray diffraction spectrum having 2θ angle positions at about 6.0, 12.1, 16.2, 17.5, 18.3, 18.7 and 19.9±0.2 degrees.

In accordance with another aspect of the present invention, there is provided a process for the preparation of saquinavir crystalline form III, which comprises:
a) stirring saquinavir in a solvent system comprising an alcohol, water and acetone; and
b) isolating saquinavir crystalline form III.

In accordance with another aspect of the present invention, there is provided novel amorphous form of saquinavir.

In accordance with another aspect of the present invention, there is provided a process for the preparation of saquinavir amorphous form, which comprises removing the solvent from a solution of saquinavir in chloroform.

In accordance with another aspect of the present invention, there is provided a process for preparing saquinavir crystalline form I which comprises:
a) dissolving saquinavir crystalline form II or III in an organic solvent;
b) optionally, removing the solvent partially or completely from the solution obtained in step (a);
c) adding a solvent selected from hydrocarbon solvents to the mass obtained in step (b); and
d) isolating saquinavir crystalline form I.

In accordance with another aspect of the present invention, there is provided a pharmaceutical composition comprising a polymorphic form of saquinavir selected from form II, form III and amorphous form or a mixture thereof; and a pharmaceutically acceptable excipient.

### DETAILED DESCRIPTION OF THE INVENTION

In accordance with one aspect of the present invention, there is provided a novel crystalline form of saquinavir designated as form II characterized by peaks in the powder x-ray diffraction spectrum having 2θ angle positions at about 6.6, 11.3, 17.1, 18.3, 20.7 and 23.5±0.2 degrees. The powdered x-ray diffractogram (PXRD) of saquinavir crystalline form II is shown in FIG. 1.

In accordance with another aspect of the present invention, there is provided a process for the preparation of saquinavir crystalline form II, which comprises:
a) stirring saquinavir in a solvent system comprising an alcohol and water; and optionally in the presence of acetonitrile; and
b) isolating saquinavir crystalline form II.

The alcohol solvent used in step (a) may be a solvent or mixture of solvents selected from the group consisting of a methanol, ethanol, isopropyl alcohol, tert-butyl alcohol and n-butyl alcohol. Preferable alcohol solvent is selected from methanol and ethanol, still more preferable alcohol solvent is methanol.

Saquinavir crystalline form II typically has a water content of above 2.0% by weight and dried product has the water content of 2.0% to 4.0%.

According to the processes described in the prior art, chromatographic purifications were required for obtaining saquinavir or a pharmaceutically acceptable salt of saquinavir such as saquinavir mesylate in pure form as measured by High performance liquid chromatographic (HPLC) method. Otherwise, many crystallizations were required to obtain saquinavir or saquinavir mesylate in pure form resulting in a loss of yield. Specifically, the impurity resulting at relative retention time (RRT) of about 1.26 with reference to saquinavir is difficult to remove. We have developed a simple process that can be used to purify saquinavir.

Thus, in accordance with another aspect of the present invention, there is provided a process for purification of saquinavir which comprises:
a) stirring saquinavir in a solvent system comprising an alcohol, water and acetonitrile; and
b) isolating saquinavir to obtain substantially pure saquinavir.

The alcohol solvent used in step (a) may be a solvent or mixture of solvents selected from the group consisting of a methanol, ethanol, isopropyl alcohol, tert-butyl alcohol and n-butyl alcohol. Preferable alcohol solvent is selected from methanol and ethanol, still more preferable alcohol solvent is methanol.

The term “substantially pure saquinavir” refers to saquinavir having purity not less than 96% as measured by HPLC. Specifically saquinavir having the impurity at about 1.26 RRT of less than 1.0% is obtained.

According to the process of present invention, saquinavir can be obtained in a purity of not less than 99% and the impurity at RRT of about 1.26 in less than 0.6%

Thus, the process of the invention may be used to obtain saquinavir in known crystalline form I. A pharmaceutically acceptable salt such as saquinavir mesylate in pure form may be prepared from pure saquinavir as obtained by the process of the invention.

In accordance with another aspect of the present invention, there is provided a novel crystalline form of saquinavir designated as form III characterized by peaks in the powder x-ray diffraction spectrum having 2θ angle positions at about 6.0, 12.1, 16.2, 17.5, 18.3, 18.7 and 19.9±0.2 degrees. The powdered x-ray diffractogram (PXRD) of saquinavir crystalline form III is shown in FIG. 2.

In accordance with another aspect of the present invention, there is provided a process for the preparation of saquinavir crystalline form III, which comprises:
a) stirring saquinavir in a solvent system comprising an alcohol, water and acetone; and
b) isolating saquinavir crystalline form III.

The alcohol solvent used in step (a) may be a solvent or mixture of solvents selected from the group consisting of a methanol, ethanol, isopropyl alcohol, tert-butyl alcohol and n-butyl alcohol. Preferable alcohol solvent is selected from methanol and ethanol, still more preferable alcohol solvent is methanol.

In accordance with another aspect of the present invention, there is provided novel amorphous form of saquinavir. The powdered x-ray diffractogram (PXRD) of saquinavir amorphous form is shown in FIG. 3.

In accordance with another aspect of the present invention, there is provided a process for the preparation of saquinavir amorphous form, which comprises removing the solvent from a solution of saquinavir in chloroform.

Preferably, the chloroform solvent is removed by methods such as distillation of the solvent at elevated temperatures, spray drying or freeze drying.

In accordance with another aspect of the present invention, there is provided a process for preparing saquinavir crystalline form I which comprises:
a) dissolving saquinavir crystalline form II or III in an organic solvent;
b) optionally, removing the solvent partially or completely from the solution obtained in step (a);
c) adding a solvent selected from hydrocarbon solvents to the mass obtained in step (b); and
d) isolating saquinavir crystalline form I.

The organic solvent used in step (a) may be selected from the group consisting of a dichloromethane, dichloroethane and chloroform. Preferable organic solvent is dichloromethane.

The hydrocarbon solvent used in step (c) may be selected from the group consisting of a hexane, cyclohexane, heptane and toluene. Preferable hydrocarbon solvent is hexane.

The saquinavir crystalline form I obtained by the process described above is characterized by peaks in the powder X-ray diffraction pattern having 2θ angle positions at about 4.7, 5.3, 9.2, 16.1 and 18.1±0.2 degrees. The typical X-ray powder diffraction pattern is shown in FIG. 4.

The novel forms of the inventions, namely saquinavir crystalline form II, form III and amorphous form are also useful intermediates for obtaining the known crystalline form I and a pharmaceutically acceptable salts of saquinavir such as saquinavir mesylate

In accordance with another aspect of the present invention, there is provided a pharmaceutical composition comprising a polymorphic form of saquinavir selected from form II, form III and amorphous form or a mixture thereof; and a pharmaceutically acceptable excipient.

Preferable pharmaceutical composition is used in an oral dosage form.

### BRIEF DESCRIPTION OF THE DRAWING

FIG. 1 is X-ray powder diffraction spectrum of saquinavir crystalline form II.

FIG. 2 is X-ray powder diffraction spectrum of saquinavir crystalline form III.

FIG. 3 is X-ray powder diffraction spectrum of saquinavir amorphous form.

FIG. 4 is X-ray powder diffraction spectrum of saquinavir crystalline form I.

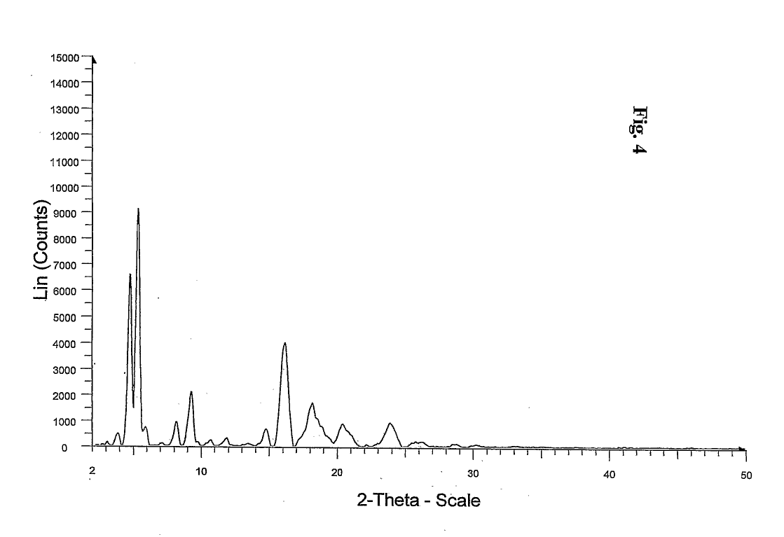

X-ray powder diffraction spectrum was measured on a bruker axs D8 advance X-ray powder diffractometer having a copper-Kα radiation. Approximately 1 gm of sample was gently flattered on a sample holder and scanned from 2 to 50 degrees two-theta, at 0.03 degrees to theta per step and a step of 0.5 seconds. The sample was simply placed on the sample holder. The sample was rotated at 30 rpm at a voltage 40 KV and current 35 mA.

Purity of saquinavir is measured by HPLC method as described for saquinavir mesylate in the US pharmacopoeia 32, Volume 3, Page 3537.

The invention will now be further described by the following examples, which are illustrative rather than limiting.

### EXAMPLES

### Example 1

N²-(2-quinolinylcarbonyl)-L-asparagine (93 gm) was suspended in dichloromethane (1000 ml), the suspension was cooled to 0-5° C. and then triethylamine (32 gm) was added. To the solution, 2,6-lutidine (5 gm) was, added, immediately pivalolyl chloride (36 gm) was added under stirring and stirred for minutes. [3S,4aS,8aS]-2-(3S-amino-2R-hydroxy-4-phenylbutyl)-N-(1,1-dimethylethyl)-decahydro-3-isoquinoline carboxamide (100 gm) was added to the reaction mass, stirred for 30 minutes, dichloromethane (400 ml) and water (400 ml) was added to the solution and layers were separated. The pH of the organic layer was adjusted to 7.0-8.0 with 10% aqueous sodium bicarbonate solution (200 ml) at 20 deg C., layers were separated. The resulting organic layer was concentrated at below 55 deg C. under vacuum pressure until solvent was distilled off. To the residue obtained was added methanol (500 ml) and stirred for 1 hour at room temperature. To the reaction mass was added water (300 ml), stirred for 30 minutes at room temperature, acetonitrile (200 ml) was added to the reaction mass and stirred for 1 hour at room temperature. Water (200 ml) was added to the reaction mass, stirred for 2 hours at room temperature, cooled to 5-10 deg C. and again stirred for 1 hour at 5-10 deg C., filtered. The solid obtained was washed with a mixture of methanol, water and acetonitrile and then dried the solid at 60-65 deg C. for 6 hours to yield 125 gm of saquinavir crystalline form II (HPLC purity: 99.5%, Impurity at 1.26 RRT: 0.06%, Water content: 2.6%).

### Example 2

Saquinavir crystalline form II (50 gm) obtained as in example 1 was dissolved in methanol (250 ml), stirred for 30 minutes at reflux, filtered on hyflow bed. Methane sulfonic acid (7.5 gm) was added, stirred for 10 hours at room temperature. The reaction mass was stirred for 1 hour at 10-15 deg C. and filtered to obtain wet solid. The wet solid obtained was dissolved in methanol (200 ml) and stirred for 30 minutes at reflux, then for 3 hours at room temperature, and then for 1 hour at 5-10 deg C. and filtered. The solid obtained was dried to yield 57 gm of saquinavir mesylate (HPLC purity: 99.5%, Impurity at 1.26 RRT: Not detected).

### Example 3

The mixture of saquinavir mesylate (100 gm, HPLC Purity: 98%, Impurity at 1.26 RRT: 0.5%), chloroform (1000 ml) and water (600 ml) was heated to 30-35 deg C. and the pH was adjusted to 11.0-11.5 with aqueous sodium hydroxide (1:1), stirred for 10 minutes and the layers were separated. The aqueous layer was extracted twice with chloroform (2×300 ml), the total organic layer was washed with water (600 ml) at 30-35 deg C. and dried with sodium bisulfate. The organic layer was passed on hi-flo bed and the solvent was distilled off under vacuum. Methanol (500 ml) was added to the residue obtained and stirred for 10 minutes at 40 deg C. The mass was stirred for 1 hour at room temperature, water (300 ml) was added and stirred for 1 hour at room temperature. To the reaction mass was added acetonitrile (200 ml) and stirred for 1 hour at room temperature, and then for 1 hour at 0-5 deg C., filtered. The solid obtained was washed with mixture of methanol, water and acetonitrile and then dried the solid at 60-65 deg C. for 7 hours to obtain 82 gm of saquinavir crystalline form II (HPLC purity: 99.5%, Impurity at 1.26 RRT: 0.05%, Water content: 2.8%).

### Example 4

Water (350 ml) was added to the mixture of saquinavir mesylate (60 gm) and dichloromethane (650 ml) was heated to 30-35 deg C. and the pH was adjusted to 11.0-11.5 with aqueous sodium hydroxide (1:1), stirred for 10 minutes and the layers were separated. The aqueous layer was extracted twice with dichloromethane (2×200 ml), the total organic layer was washed with water (300 ml) at 30-35 deg C. and dried with sodium bisulfate. The organic layer was passed on hi-flo bed and the solvent was distilled off under vacuum. Methanol (300 ml) was added to the residue obtained and stirred for 10 minutes at 40 deg C. The mass was stirred for 1 hour at room temperature, water (200 ml) was added and stirred for 1 hour at room temperature, and then for 1 hour at 0-5 deg C., filtered. The solid obtained was washed with mixture of methanol and water and then dried the solid at 60-65 deg C. for 7 hours to obtain 48 gm of saquinavir crystalline form II.

### Example 5

Saquinavir crystalline form II (100 gm) obtained as in example 3 was dissolved in dichloromethane (2250 ml) at 40 deg C., stirred for 30 minutes at 40 deg C. and distilled off the solvent under vacuum to obtain residue. To the residue was added hexane (1000 ml) and cooled to room temperature, filtered, washed with hexane and dried at 60-65 deg C. for 6 hours to obtain 97 gm of saquinavir crystalline form I.

### Example 6

Example 5 was repeated using saquinavir crystalline form III instead of saquinavir crystalline form II to obtain saquinavir crystalline form I.

### Example 7

Water (300 ml) was added to the mixture of saquinavir mesylate (50 gm) and dichloromethane (600 ml) was heated to 30-35 deg C. and the pH was adjusted to 11.0-11.5 with aqueous sodium hydroxide (1:1), stirred for 10 and the layers were separated. The aqueous layer was extracted twice with dichlorromethane (2×200 ml), the total organic layer was washed with water (300 ml) at 30-35 deg C. and dried with sodium bisulfate. The organic layer was passed on hi-flo bed and the solvent was distilled off under vacuum. Methanol (250 ml) was added to the residue obtained and stirred for 10 minutes at 40 deg C. The mass was stirred for 1 hour at room temperature, water (160 ml) was added and stirred for 1 hour at room temperature. To the reaction mass was added acetone (120 ml) and stirred for 1 hour at room temperature, and then for 1 hour at 0-5 deg C., filtered. The solid obtained was washed with mixture of methanol, water and acetone and then dried the solid at 60-65 deg C. for 7 hours to obtain 40 gm of saquinavir crystalline form III.

### Example 8

The mixture of saquinavir mesylate (100 gm), chloroform (1000 ml) and water (600 ml) was heated to 30-35 deg C. and the pH was adjusted to 11.0-11.5 with aqueous sodium hydroxide (1:1), stirred for 10 minutes. Then the layers were separated, the aqueous layer was extracted twice with chloroform (300 ml), and the total organic layer was washed with water (600 ml) at 30-35 deg C. and dried with sodium bisulfate. The organic layer was passed on hi-flo bed and the solvent distilled off under vacuum to obtain saquinavir amorphous form. Cyclohexane (100 ml) was added to the saquinavir amorphous form obtained and the solvent distilled off completely under vacuum at below 60 deg C. To the residue was added cyclohexane (800 ml), stirred for 2 hours at room temperature, filtered and dried at 60-65 deg C. for 8 hours to obtain 84 gm of saquinavir amorphous form.

We claim:

1. A saquinavir crystalline form II, characterized by an X-ray powder diffractogram having peaks expressed as 2θ angle positions at about 6.6, 11.3, 17.1, 18.3, 20.7 and 23.5±0.2 degrees.

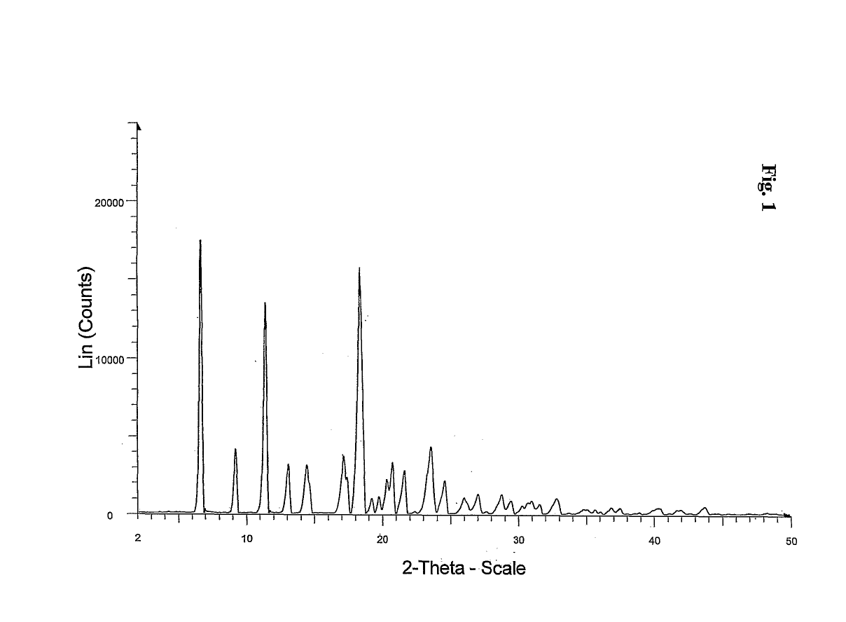

2. A saquinavir crystalline form II, characterized by an x-ray powder diffractogram as shown in FIG. 1.

3. A process for the preparation of saquinavir crystalline form II as defined in claim 1, which comprises:
a. stirring saquinavir in a solvent system comprising an alcohol and water; and optionally in the presence of acetonitrile; and
b. isolating saquinavir crystalline form II.

4. The process as claimed in claim 3, wherein the alcohol solvent used in step (a) is a solvent or mixture of solvents selected from methanol, ethanol, isopropyl alcohol, tert-butyl alcohol and n-butyl alcohol.

5. The process as claimed in claim 4, wherein the alcohol solvent used in step (a) is selected from methanol and ethanol.

6. The process as claimed in claim 5, wherein the alcohol solvent used in step (a) is methanol.

7. A process for purification of saquinavir, which comprises:
a. stirring saquinavir in a solvent system comprising an alcohol, water and acetonitrile; and
b. isolating saquinavir to obtain substantially pure saquinavir.

8. The process as claimed in claim 7, wherein the alcohol solvent used in step (a) is a solvent or mixture of solvents selected from methanol, ethanol, isopropyl alcohol, tert-butyl alcohol and n-butyl alcohol.

9. The process as claimed in claim 8, wherein the alcohol solvent used in step (a) is selected from methanol and ethanol.

10. The process as claimed in claim 9, wherein the alcohol solvent used in step (a) is methanol.

11. A saquinavir crystalline form III, characterized by an X-ray powder diffractogram having peaks expressed as 2θ angle positions at about 6.0, 12.1, 16.2, 17.5, 18.3, 18.7 and 19.9±0.2 degrees.

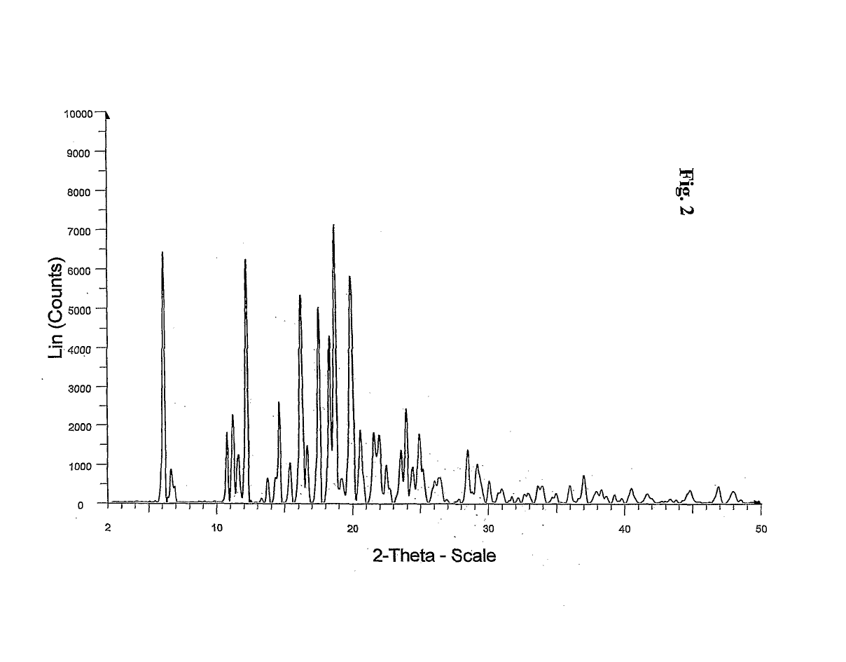

12. A saquinavir crystalline form III, characterized by an x-ray powder diffractogram as shown in FIG. 2.

13. A process for the preparation of saquinavir crystalline form III as defined in claim 11, which comprises:
a. stirring saquinavir in a solvent system comprising an alcohol, water and acetone; and
b. isolating saquinavir crystalline form III.

14. The process as claimed in claim 13, wherein the alcohol solvent used in step (a) is a solvent or mixture of solvents selected from methanol, ethanol, isopropyl alcohol, tert-butyl alcohol and n-butyl alcohol.

15. The process as claimed in claim 14, wherein the alcohol solvent used in step (a) is selected from methanol and ethanol.

16. The process as claimed in claim 15, wherein the alcohol solvent used in step (a) is methanol.

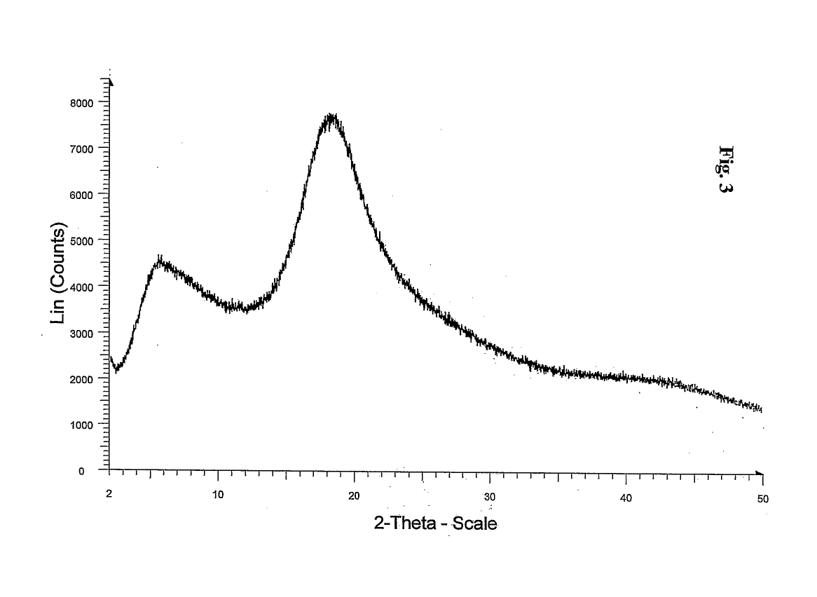

17. A saquinavir amorphous form, characterized by an x-ray powder diffractogram as shown in FIG. 3.

18. A process for the preparation of saquinavir amorphous form as defined in claim 17, which comprises removing the solvent from a solution of saquinavir in chloroform.

19. A process for the preparation of saquinavir crystalline form I, which comprises:
a. dissolving saquinavir crystalline form II or III in an organic solvent;
b. optionally, removing the solvent partially or completely from the solution obtained in step (a);
c. adding a solvent selected from hydrocarbon solvents to the mass obtained in step (b); and
d. isolating saquinavir crystalline form I.

20. The process as claimed in claim 19, wherein the organic solvent used in step (a) is selected from dichloromethane, dichloroethane and chloroform.

21. The process as claimed in claim 20, wherein the organic solvent used in step (a) is dichloromethane.

22. The process as claimed in claim 19, wherein the hydrocarbon solvent used in step (c) is selected from hexane, cyclohexane, heptane and toluene.

23. The process as claimed in claim 22, wherein the hydrocarbon solvent used in step (c) is hexane.

24. A pharmaceutical composition comprising a polymorphic form of saquinavir selected from form II, form III and amorphous form or a mixture thereof and a pharmaceutically acceptable excipient.

25. The pharmaceutical composition as claimed in claim 24, wherein the pharmaceutical composition is used in a oral dosage form.

26. The pharmaceutical composition as claimed in claim 25, wherein the oral pharmaceutical dosage forms is a tablet or a capsule.
